(19) United States
(12) Patent Application Publication
(10) Pub. No.: US 2018/0221611 A1
(43) Pub. Date: Aug. 9, 2018

(54) BETTER BITE BLOCK

(71) Applicant: Terence Gilhuly, Vancouver (CA)

(72) Inventor: Terence Gilhuly, Vancouver (CA)

(21) Appl. No.: 15/888,073

(22) Filed: Feb. 4, 2018

Related U.S. Application Data
(60) Provisional application No. 62/454,861, filed on Feb. 5, 2017.

(51) Int. Cl. A61M 16/04

(52) U.S. Cl. CPC A61M 16/0493; A61M 2205/0216

(57) ABSTRACT

Patients undergoing general anesthetics frequently experience emergence clenching where the patient may bite onto their breathing tube and may clench their teeth. These activities lead to dental damage and negative pressure pulmonary edema. The invention of this patent is a simpler, better bite block that may be used to prevent these injuries. The bite block presented comprises a deformable material used as a core around which a bite-resistant material is wrapped to prevent damage to the core by patient biting. The bite-resistant material may be formed into a distal tail that the user can pull to retain or retrieve the device. It may also have a patch of adhesive to attach the bite block to locations including the patient, the surgical drapes and the patient's breathing airway, and thereby prevent loss of the bite block.

### CROSS-REFERENCE TO RELATED APPLICATIONS

This application draws from Provisional application 62/454,861, titled “A BETTER BITE BLOCK”, of date Feb. 5, 2017.

### BACKGROUND

Patients emerging from anesthesia tend to try to close their mouth and clench their teeth, often biting on their airway tube in the process. This is known as emergence clenching and it may lead to problems including dental and soft tissue damage; negative pressure pulmonary oedema; and reduced oxygenation (hypoxia and hypercarbia) of the patient due to an obstructed or damaged endotracheal tube.

Negative pressure pulmonary edema (NPPE) is a “dangerous and potentially fatal condition with a multifactorial pathogenesis. Frequently, NPPE is a manifestation of upper airway obstruction, the large negative intrathoracic pressure generated by forced inspiration against an obstructed airway is thought to be the principal mechanism involved. This negative pressure leads to an increase in pulmonary vascular volume and pulmonary capillary transmural pressure, creating a risk of disruption of the alveolar-capillary membrane” [1].

An estimate of the severity of NPPE comes from [2]: “The sequelae of acute NPPE can be devastating with mortality as high as 11-40%. NPPE can complicate the clinical course and outcome of otherwise healthy patients undergoing simple surgical procedures. Requirement for prolonged endotracheal intubation and intensive care is not uncommon.” Consequences of NPPE include further treatment in the ICU for upwards of 8 days [5], [6].

The incidence of NPPE has been reported to be 0.05%-0.1% of all anesthetic practices, and may develop in 11% of patients requiring active intervention for acute upper airway obstruction [1]. In [3], the incidence of NPPE was estimated to be up to 12%, but may be higher because of a lack of familiarity with the syndrome. All causes of obstructed upper airway may lead to NPPE with laryngospasm during intubation or post-anesthesia being the most common [3].

Dental trauma includes such injuries as broken or chipped teeth. The reported incidence of dental trauma has been estimated between 0.04% and 0.05% of individuals undergoing a general anaesthetic risk dental damage [4].

One approach that partially mitigates these problems is to use an oropharyngeal airway about the endotracheal tube. Oropharyngeal airways surround and can thereby be used to protect the endotracheal tube from biting by teeth or gingiva. Oropharyngeal airways are typically made of a hard plastic and are of a significant size relative to the oral cavity. Because of their shape and composition, the presence of these airways can lead to other injury, including lip and tongue edema, bleeding, hoarseness, sore throat, macroglossia and ischemic myonecroses of the muscles of the tongue.

Another approach to mitigating clenching is the use of bite blocks. Bite blocks typically comprise material interposed between the patient's teeth, usually the molars or incisors, to prevent the teeth from coming together during emergence. Manufacturers of some commercially available devices and their basic description include the following:
a. South Medic's Endotracheal Tube Protector forms a barrier around the tube that the patient can bite on. It has a safety ring to attach it to the endotracheal tube.
b. The Ethox Bite Block is a tunnel through which the endotracheal tube is placed.
c. Richmond Dental makes the Soft Bite Block, which is a dense cylindrical cotton roll wrapped in silky yarn.
d. B and B makes bite blocks comprised of plastic split tubes to surround the endotracheal tube and then are either affixed to the patient with an adhesive tab also stuck to the bite block, or to the endotracheal tube with a zapstrap.
e. Teleflex's BiteGard Bite Block is a disposable, compressible molar bite block that appears to be a thin piece of plastic intended to be placed between the patient's molars.

### REFERENCES

[1] Balu Bhaskar and John F. Fraser, “Negative pressure pulmonary edema revisited: Pathophysiology and review of management”. Saudi J Anaesth. 2011 July-September; 5(3): 308-313.
[2] Website: http://www.openanesthesia.org/negative_pressure_pulmonary_edema_physiology/
[3] Malcolm Lemyze and Jihad Mallat, “Understanding negative pressure pulmonary edema”. Intensive Care Med. 2014; 40(8): 1140-1143.
[4] Jeremy Windsor and Jane Lockie. “Anaesthesia and dental trauma”. Anaesthesia and Intensive Care Medicine, 9:8 355ff
[5] Tam Dieu MD and Edward Upjohn MD. “Negative Pressure Pulmonary Edema in Healthy Cosmetic Surgery Patients.” Aesthetic Surgery Journal, Volume 23, Issue 4, pp. 270-273.
[6] Vishvesh M. Mehta, MD; Gady Har-El, MD; and Nira A. Goldstein, MD. “Postobstructive Pulmonary Edema After Laryngospasm in the Otolaryngology Patient”. The Laryngoscope

### PATENT DOCUMENTS

See U.S. Pat. Nos. 4,351,331, 5,305,742, 5,469,865, 5,513,633, 5,533,524, 5,655,519, 6,067,985, 6,526,978, 6,533,761, 6,755,191, 6,890,322, 7,066,735, 7,963,286, 7,975,695, 8,096,300, 8,146,601, 8,251,069, 8,302,597, 8,636,008, 8,783,263 and 9,233,221; and US applications 20020095118, 20020095119, 20100006110, 20100236548 and 20130118507.

### BRIEF SUMMARY

The incidence of damage due to emergence clenching may be so common due to the lack of use of bite blocks. This in turn may be due to failings of the currently available bite blocks. Problems with currently available bite blocks include:
a. Complexity: extra features for strapping complicate use. This limits usage.
b. Cost: often the costs can be excessive due to the demands of the extra complexity. Cost also limits the usage of the devices.
c. Hardness: if the device is too hard, the patient is at greater risk of tooth damage, particularly for those patients who have had dental work done.
d. Softness/compressibility: if the device is too soft, the device must be made thick to compensate making it obtrusive and/or being soft may not prevent clenching from occluding tubes in the patient's mouth.
e. Size: thinner bite blocks may only reduce damage to the teeth and may not prevent occlusion of the endotracheal tube.
f. Time consuming: Loss of the bite block within the oral cavity can cause airway obstruction. Therefore a means of preventing bite block loss such as by fixing the bite block to the patient and/or surgical drapes and/or airways is required. However, preparation of the retrieval and/or fixation means can be time-consuming, such as when sutures are required to fixate the bite block.
g. Inconvenience: some bite blocks with security features that encircle the intubation tube can only be placed prior to intubation. Being placed prior to intubation means the device will be present throughout the procedure and can be an impediment to the anesthesiologist's work throughout the procedure, as opposed to being placed at the end of the procedure when it is useful and needed.

The invention is a simple device for reducing dental and soft tissue damage, negative pressure pulmonary oedema, and reduced oxygenation (hypoxia and hypercarbia) of the patient due to emergence clenching. This invention attempts to correct the defects of other bite blocks in the field. By making a better and more convenient to use bite block, this invention attempts to make the bite block a more frequently used device and thereby further decrease the impact of emergence clenching.

A first form of the invention comprises a deformable material protected with a bite resistant covering to be inserted between the patient's teeth. Should the patient attempt to bite on the tube, the deformable material will be compressible enough so that the patient's teeth are not damaged and firm enough that the tube being protected cannot be occluded.

A second form of the invention provides a tail for retrieval of the device.

A third form of the invention further includes adhesive coating on one or more portions of the protective material's tail such that the invention may be attached to the patient, their drapes, their airway and/or another convenient location with this adhesive.

A fourth form of the invention includes a deformable material composed of different multiple materials.

A fifth form of the invention features multiple tails with adhesive patches to allow fixation of the device to more than one location on the patient, including their body (primarily their face), their drapes and their airway circuit.

### BRIEF DESCRIPTION OF THE DRAWINGS

FIG. 1 illustrates a bite block in a perspective view.

FIG. 2 illustrates a second bite block with a tail in a perspective view.

FIG. 3 illustrates a third bite block with a tail and a liner covering exposed adhesive on the tail in a perspective view.

FIG. 4 illustrates a side view of the third bite block with a tail and a liner covering exposed adhesive on said tail.

FIG. 5 shows an exploded view of the third bite block.

FIG. 6 illustrates a side view of the third bite block with a tail and a liner covering exposed adhesive on said tail, and with deformable material constructed of more than one material.

FIG. 7 illustrates a fourth bite block with multiple tails.

### DETAILED DESCRIPTION

In this section, a detailed description of the bite block of this invention is presented.

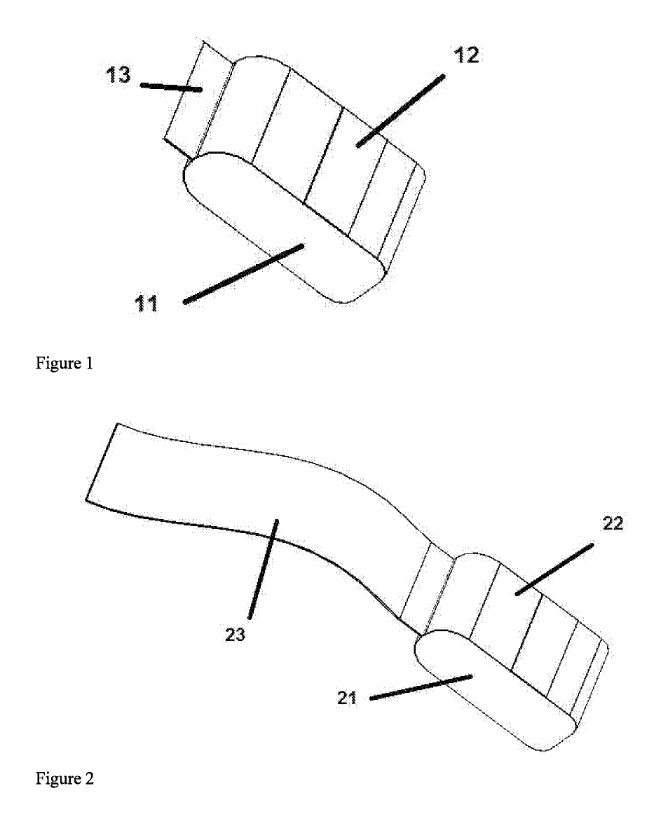

In FIG. 1 a first instance of the invention is shown. There, the invention comprises a deformable material 11 protected with bite resistant protective covering 12 sealed around deformable material 11 at protective covering joint 13.

Deformable material 11 can comprise a foam or rubber block, preferable of one or more biocompatible materials, and preferably the materials are non-absorptive of body fluids such as saliva and blood, and also water and saline. The block is ideally of rectangular construction and shaped to be large enough for handling but not so large as to interfere with the endotracheal tube or any necessary procedures or manipulation. The block's thickness can be set so the block can fit in a patient's mouth but be of size greater than a typical endotracheal tube diameter; a range from at least 0.25 and at most 1.5 inches may be suitable.

Deformable material 11 ideally is somewhat compressible so that no damage to the teeth occurs on biting, but not so compressible that the endotracheal tube can be occluded by biting. For example, a bite block of this invention that is 20 mm thick protecting an endotracheal tube 10 mm in diameter should be soft enough to compress partially but stiff enough to resist compression to 50% of its thickness at pressures between 68 psi and 150 psi, which correspond to the normal range of forces for chewing and clenching (http://www.dental-health.com/biteocclusionpohl.html).

Should deformable material 11 be constructed from foam, ideally the foam is of closed-cell formation so as not to become waterlogged with secretions. Absorption of fluids leads to a decrease in performance of a foam or sponge when being chewed or clenched. This is particularly important in instantiations of the invention where surfaces of deformable material 11 are not completely covered by protective covering 12, as would happen if protective covering 12 was a wrapped tape covering only four sides of six to protect the biting surfaces only.

Deformable material 11 may be shaped with rounded corners to present better aesthetics and patient comfort.

Protective covering 12 is preferable made of a bite resistant material, and is intended to surround deformable material 11 and will be subject to biting. Protective covering 12 prevents damage to deformable material 11 by preventing damage due to patient biting and maintaining the integrity of deformable material 11. The protective covering prevents pieces of the bite block from being broken or bitten off and ingested.

As the biting forces of human incisors can be as much as 15.3 kg https://www.scribd.com/document/33955152/Bite-Forces-and-Bite-Pressure), to be bite resistant the material should be able to resist puncture and tearing from similar forces.

In a preferred instantiation of the invention, protective material 12 can comprise one or more pieces of bite resistant tape where at least one side of the tape is covered at least partially with an adhesive capable to adhering to deformable material 11 and itself. Preferably protective material 12 is a single continuous piece to minimize possibility of fraying and disassembly. The tape is preferably a silk or silk-synthetic tape, such as 3M Duropore line of tapes, which are known to resist biting.

For construction, protective covering 12 is wrapped around or otherwise caused to surround deformable material 11 covering at least the surfaces that may come into contact with the patient's teeth to protect deformable material 11. In the preferred instantiation where protective material 12 is a single continuous piece of adhesive coated tape, protective covering 12 joins to itself at protective covering joint 13.

In another instantiation of the invention where protective covering 12 is not an adhesive coated tape, the protective covering 12 is wrapped about deformable material 11 and secured through other means including one or more of heat sealing; RF, friction or ultrasonic welding; gluing, sewing and stapling.

In another aspect of this invention, protective covering 12 can be a shrink wrap material preferably in a tubular construction so that deformable material 11 can be inserted inside the tube of protective covering 12 and sealed in place with heat.

FIG. 2 illustrates a second instantiation of the invention, a bite block with a tail. Again, the invention comprises a deformable material 21 with protective covering 22 as described in the previous instantiations of FIG. 1. In this aspect of the invention, tail 23 is an extension added to the device that the physician using the bite block can use to retrieve the bite block should it slip out of position. Tail 23 should be grippable should the device fall into the patient's mouth. A length of 3 inches or more allows for some of the tail to stick out of the average patient's mouth where it can be safely grasped without fear of biting fingers, while the bulk of the device is in the patient's oral cavity.

In an ideal instantiation of the invention, tail 23 is a continuation of protective covering 22. Tail 23 can comprise a length of material longer than the perimeter dimension of deformable material 21 that remains after wrapping around deformable material 21, and is then folded onto itself. Using one continuous material in this fashion allows for simple and cost-effective construction and supply chain management. For the instantiation where protective covering 22 is an at least partially adhesive coated tape, protective covering tail 23 is formed as the two pieces of tape come together. For the instantiation where protective covering 22 is made from heat shrink or other type of tubing, protective covering tail 23 can be formed from a length of material not needed to contain deformable material 21.

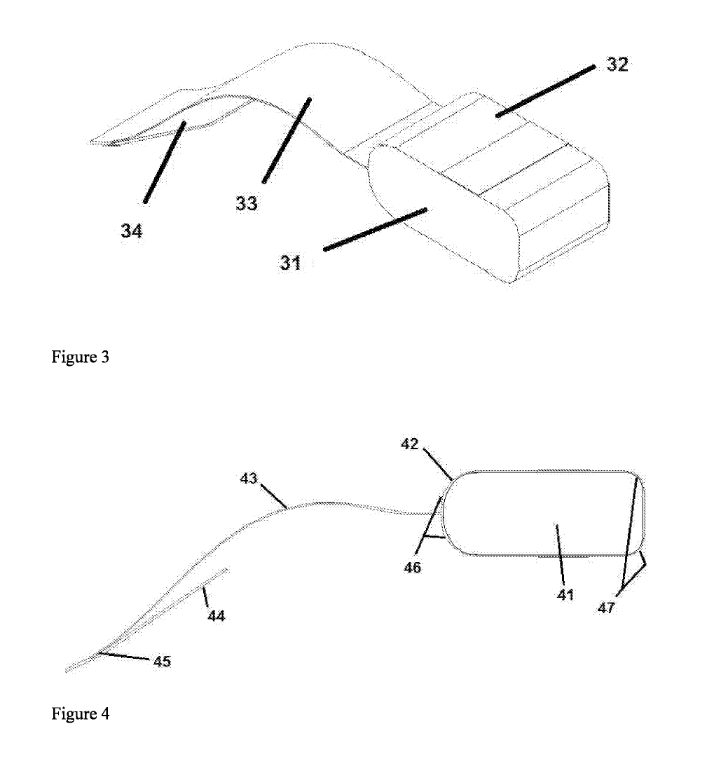

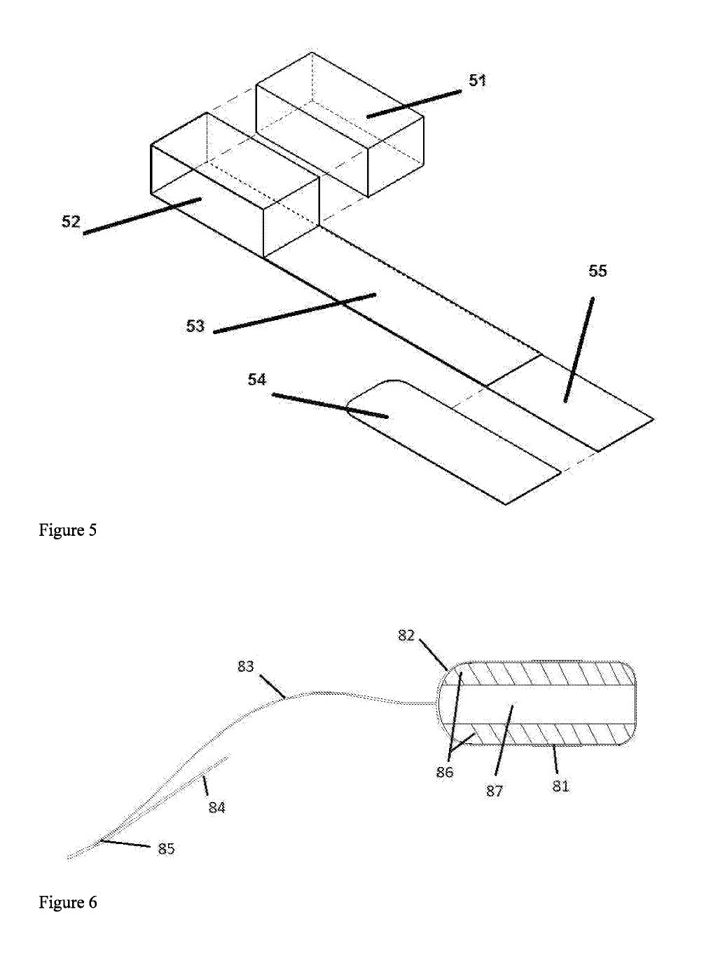

A third instantiation of the invention is shown in FIG. 3 in a perspective view, in FIG. 4 in a side view and in FIG. 5 in an exploded view. An adhesive (adhesive 45 underneath adhesive covering liner 44 in FIG. 4 and shown with liner removed as exposed adhesive 55 in FIG. 5) is provided on one or more areas of protective covering tail 33 to provide a means of attaching the bite block to an anchor including the patient and/or to their airway and/or to surgical drapes. In FIGS. 3 and 4, the bite block with a tail further comprises adhesive covering liner 34 (also as adhesive covering liner 44 in FIG. 4 and adhesive covering liner 54 in FIG. 5) which covers adhesive 45 to protect adhesive 45 from fowling before it is used.

Adhesive 45 is ideally biocompatible for patient contact, and can be chemically and mechanically able to adhere to protective covering tail 43, surgical drapes, the patient endotracheal tube and airway.

In instantiations where protective covering 42 is a tape, adhesive 45 may be constructed by coating a surface of protective coating tail 43 with a suitable adhesive. Preferably adhesive 45 is placed at the distal end of protective coating tail 43 to give more range of attachment. Also, adhesive 45 may be provided for a bite block with protective covering tail 43 formed of an adhesive covered tape by making one end of the tape used to construct protective covering tail 43 shorter than the other such that a portion of the adhesive on the tape remains exposed after the two ends of tape are folded onto each other.

Adhesive covering liner 44 ideally is of larger area than adhesive 45 so that adhesive covering liner 44 can cover adhesive 45 and have material not covering adhesive 45 that may be used as a pull-tab to remove adhesive covering liner 45 when the user wants to attach the bite block.

Adhesive covering liner 44 can be comprised of a paper substrate with one or more sides coated with silicone or another material compatible for light hold and release from adhesive 45.

Adhesive covering liner 44 can be replicated by the bite block's container or packaging, such as if it is presented on a tray, for example.

FIG. 5 shows an exploded view of a preferred instantiation of the invention. Deformable material 51 has been removed from its pocket created by protective covering 52. Protective covering 52 is shown shaped as it would be wrapped around deformable material 51, using an adhesive coated tape for the material of protective covering 52. Exposed adhesive 55 is shown at the distal end of protective covering tail 53. Adhesive covering liner 54 is shown removed from and to the side of exposed adhesive 55.

FIG. 6 shows the bite block of FIG. 4 where deformable material 41 has been replaced by multiple material deformable material 81. Multiple material deformable material 81 is composed of a combination of differing materials, ideally differing in mechanical strength so as to have more-compressible and less-compressible materials. FIG. 6 shows an exemplary instantiation where more-compressible material 86 and less-compressible material 87 are arranged in a stack structure with faces planar to the biting surface as opposed to edge-on, and as before they are encircled by bite resistant protective material 83. With a more compressible material presented to the teeth and less compressible materials located in the core of multiple-material deformable material 81, the patient will be presented with a softer biting surface reducing the likelihood of teeth damage, and still have protection from collapsing of the endotracheal tube due to having a less compressible material at the core maintaining the thickness of the device.

The deformable material could be made of more than one material joined together by methods including gluing, hot air and friction welding, or constructed using additive manufacturing techniques such as overmolding where one material could be injection molded or lapped about a different core material. The materials could be different in chemistry, such as with a rubber and a plastic or as with a polyethylene and a polypropylene plastic; and could be different in material properties including differing densities, compressibility. Example materials include polyethylene, cross-linked polyethylene, polypropylene and polyurethane foams, expanded vinyl acetate (EVA), and thermoplastics and other elastomers of varying densities and firmness. An example material stackup is lower density polyethylene foam layers sandwiching and heat bonded to a higher density polyethylene foam layer. For reduced construction costs, a bilayer stack could also be constructed with one more-compressible layer for teeth protection and one less-compressible for endotracheal tube protection. As well, instead of a permanent joining process, multiple material deformable material 81 may be simply held together by the wrap around them of protective covering 83.

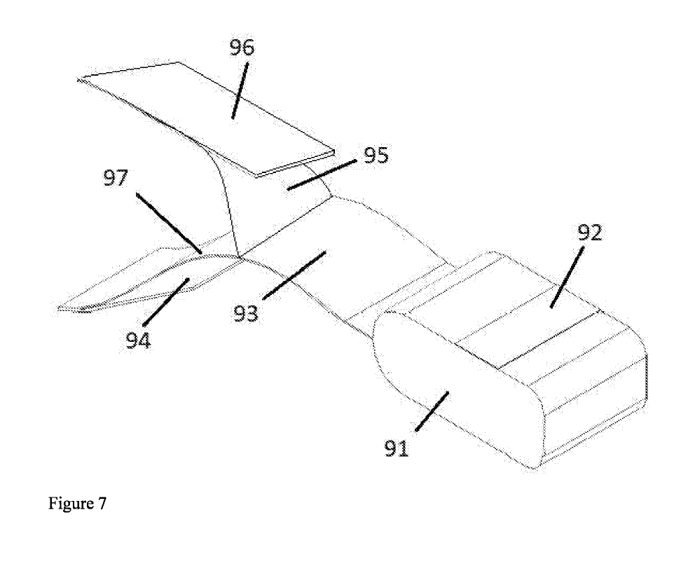

In a final instantiation of the invention for this disclosure, the invention could have more than one tail to permit attachment of the bite block to more than one location. FIG. 7 shows a two tailed bite block comprising deformable material 91, protective covering 92, and main tail 93 which splits into two sections defined by upper tail 96 and lower tail 94. Both of these tail sections have adhesive patches. Upper liner 96 covers an adhesive patch on upper tail 96 and lower liner 94 covers an adhesive pouch on lower tail 96. Methods of constructing the multiple tails include using multiple pieces of material joined together and using intricate folding of the tail.

### Exemplary Usages

In an exemplary use the device of FIG. 3 is used to prevent injury due to emergence clenching. For this example, the device's deformable material is a polyethylene foam with compressive strength of approximately 300 KPa at 10% compression. Further, the foam is 0.75 inches thick between its top and bottom sides, 2 inches long from proximal to distal sides, and 1 inch wide. Also for the example, the deformable material is wrapped with a bite-resistant protective material of silk tape with top, bottom, front and back sides of the foam covered by the tape and with enough tape to form a tail of approximately six inches in length by folding the tape over itself and thereby mating adhesive sides. The protective tape in this example is overlapped for all but two inches at the end on one side where the adhesive side is left uncovered, leaving a 2 inch by 1 inch (for an area of 2 inches square) adhesive patch and then covered by the adhesive covering liner.

The device is inserted into the patient's mouth deformable material proximal end first, and placed between the patient's molars with device top side presented to the upper teeth and device bottom side presented to the lower teeth such that the bite resistant material contacts the patient's teeth. The adhesive at the distal end of protective material tail is exposed by removing the adhesive covering liner and the exposed adhesive is pressed to the patient's chin. Pressing the adhesive to the patient removably attaches the device to the patient making it easy to remove when finished and preventing it from falling into the patient's mouth.

In another exemplary use a second bite block is placed on the opposite side of the patient's mouth so the two bite blocks provide greater crush resistance for the endotracheal tube and a more stabilized mandible.

In a third exemplary use a single bite block is placed as done in the first two examples, but it is placed between the patient's incisors.

In a fourth exemplary use the adhesive for the bite block is pressed to and the bite block is attached to the patient's breathing circuit. When the patient is extubated the bite block is removed along with the endotracheal tube.

### Terms

All terms used herein, are used in accordance with their ordinary meanings unless the context or definition clearly indicates otherwise. Also unless expressly indicated otherwise, the use of “or” includes “and” and vice-versa. Non-limiting terms are not to be construed as limiting unless expressly stated, or the context clearly indicates, otherwise (for example, “including”, “having” and “comprising” typically indicate “including without limitation”). Singular forms, including in the claims, such as “a”, “an” and “the” include the plural reference unless expressly stated, or the context clearly indicates, otherwise.

Unless stated specifically, patient refers to any biological system, human or other animal. While examples pertain more to human application, veterinary and experimental and other applications are included.

From the foregoing, it will be appreciated that, although specific embodiments have been discussed herein for purposes of illustration, various modifications may be made without deviating from the scope of the discussion herein. Accordingly, the systems and methods, etc., include such modifications as well as all permutations and combinations of the subject matter set forth herein and are not limited except as by the appended claims or other claim having adequate support in the discussion herein.

What is claimed is:

1. A device to be placed between a patient's teeth for the prevention of injury due to emergence clenching comprising:
a) A deformable material of substantially rectanguloid shape having top, bottom, distal, proximal, lingual and buccal sides,
b) A protective covering wrapped around said deformable material covering at least two sides to contact the patient's teeth, where said protective covering is resistant to damage from biting, and
c) At least one tail extension.

2. The device of claim 1 further comprising at least one adhesive patch located on said at least one tail extension.

3. The device of claim 2 further comprising at least one removable liner to cover said at least one adhesive patch.

4. The device of claim 1 where said protective covering and said at least one tail extension are made from a single piece of material.

5. The device of claim 1 where said protective covering is an adhesive coated tape.

6. The device of claim 5 where said protective covering is wrapped around said deformable material and then folded onto itself such that the distal end of said protective covering is incompletely covered to leave exposed a portion of the adhesive of said adhesive coated tape.

7. The device of claim 6 where said further comprising at least one removable liner to cover said exposed adhesive.

8. The device of claim 1 where said deformable material is composed of rubber or foam.

9. The device of claim 1 where said deformable material is composed of polystyrene, polyethylene, polypropylene, polyurethane or expanded vinyl acetate.

10. The device of claim 1 where said deformable material is non-absorptive of fluids including blood, saliva, water and saline.

11. The device of claim 1 where said deformable material comprises more than one material.

12. The device of claim 11 where said more than one material comprises at least one material that is of greater compressibility than the other said materials.

13. The device of claim 12 where said more than one material comprises a more compressible material and a less compressible material.

14. The device of claim 12 where said more than one material comprises layers where the top and bottom layers are of more compressible material and at least one internal layer is less compressible than the top and bottom materials.

15. The device of claim 1 where said deformable material has a dimension of thickness between said top and bottom sides of at least 0.25 and at most 1.5 inches.

16. The device of claim 1 where said tail has a length greater than 3 inches.

17. The device of claim 2 where said at least one adhesive patch has an area of at least 2 inches square.

18. The device of claim 1 where the deformable material compresses to 50% at pressures of between 68 and 150 psi.

19. A device to be placed between a patient's teeth for the prevention of injury due to emergence clenching comprising:
a) A deformable material of substantially rectanguloid shape having top, bottom, distal, proximal, lingual and buccal sides, and having thickness between 0.25 and 1.5 inches, comprising multiple materials where at least top and bottom sides are of a more compressible material and there is an inner core material of less compressible material;
b) A protective covering wrapped around said deformable material covering at least two sides to contact the patient's teeth, where said protective covering is resistant to damage from biting, composed of an adhesive tape;
c) A tail extension formed by said protective covering adhesive tape where said tape wraps about said deformable material on said deformable material's top, bottom, distal and proximal sides and is folded onto itself with adhesive side to adhesive side such that most of the tail is overlapped and a small section at the tip of the tail is a single layer such that the adhesive is exposed; and
d) A removable liner to cover said exposed adhesive.

20. A device to be placed between a patient's teeth for the prevention of injury due to emergence clenching comprising:
a) A deformable material of substantially rectanguloid shape having top, bottom, distal, proximal, lingual and buccal sides, and having thickness between 0.25 and 1.5 inches;
b) A protective covering wrapped around said deformable material covering at least two sides to contact the patient's teeth, where said protective covering is resistant to damage from biting, composed of an adhesive tape;
c) At least two tail extensions where each tail extension is formed by said protective covering adhesive tape where said tape covers said deformable material on said deformable material's top, bottom, distal and proximal sides, and said tape is folded onto itself with adhesive side to adhesive side such that all but a small section at the tip of each tail are double layered and the section at the tip is a single layer such that the adhesive is exposed; and
d) At least one removable liner to cover said exposed adhesive.
